(19) United States
(12) Patent Application Publication
(10) Pub. No.: US 2025/0389733 A1
(43) Pub. Date: Dec. 25, 2025

(54) Method for determining risk factors for neurodegenerative diseases in blood

(71) Applicants: Jijing WANG, Stockholm (SE); Roman ZUBAREV, Stockholm (SE)

(72) Inventors: Jijing WANG, Stockholm (SE); Roman ZUBAREV, Stockholm (SE)

(21) Appl. No.: 18/704,939

(22) PCT Filed: Oct. 29, 2021
(86) PCT No.: PCT/SE2021/051084

§ 371 Date: Apr. 26, 2024

(51) Int. Cl. G01N 33/68

(52) U.S. Cl. CPC G01N 33/6896; G01N 2800/2821; G01N 2800/50

(57) ABSTRACT

The current disclosure describes a method to differentiate whether a blood sample belongs to a normal group or a risk group considering isoAsp. The disclosed method comprises: obtaining a first set of test blood samples and a second set of blood samples that are considered belonging to a normal (control) group; obtaining plasma from said blood samples; measuring the relative abundance of anti-isoaspartate antibodies in each plasma sample; measuring the occupancy of isoaspartate residue in a representative HSA sequence location in each plasma sample; based on the distributions in the set of normal plasma samples of the relative abundances of anti-isoaspartate antibodies and of the occupancies of isoaspartate residues in a representative HSA sequence location, establishing a statistical model for the probability for a given plasma sample to be a normal sample; attributing every plasma test sample to either normal or risk group based on the maximum likelihood according to their measured values and said statistical model.

### TECHNICAL FIELD

This invention relates to medicinal chemistry. It describes a method for evaluating the risk for developing or fast progression of human neurological or cognitive diseases caused by protein aggregation, as well as for assessing the quality of human blood plasma, and its suitability for transfusion or further storage. The method is based on measuring the occupancy of isoaspartic acid residue in an abundant blood protein as well as on the abundance of antibodies against this residue in blood, and comparing the obtained values to the threshold values derived from the corresponding analysis of blood from healthy individuals.

### BACKGROUND OF THE INVENTION

Proteins are biopolymers composed of amino acid residues. In vivo, proteins have a limited lifetime. One of the main reasons for the loss of function by a protein is the loss of ammonia molecule from the asparagine (Asn) residue. The resultant cyclic succinamide intermediate is unstable and quickly attaches a water molecule, which leads to formation of the unnatural amino acid residue isoaspartic acid (isoAsp, FIG. 1). This process is known as Asn deamidation. Another mechanism of isoAsp formation is the isomerisation of the normal L-aspartic acid (Asp) residue. The deamidated proteins with isoAsp lose not only their function, but also their structure and become prone to aggregation. The enzyme Protein Isoaspartate MethylTransferase (PIMT) repairs deamidated proteins by methylation of the isoAsp residues using the small molecule called S-adenosyl methionine (SAM) and converting it to S-adenosyl homocysteine (SAH). Methylated isoAsp eventually loses methanol molecule and converts to succinimide intermediate which, upon water attachment, converts with some probability to a normal Asp residue. This process largely restores protein structure and function, and it occurs mostly in liver.

However, with age the repair function of liver declines, while the demand for repair increases, which leads to the deficit of repair and results in isoAsp accumulation in blood proteins. The isoAsp-containing proteins aggregate themselves and trigger aggregation of other proteins. There is experimental evidence that this process ultimately lead to amyloidosis, that is formation of amyloid oligomers and fibrils, which causes oxidative stress, inflammation and other pathological processes that contribute to the development of Alzheimer's Disease (AD) and similar neurological disorders (Yang H, Lyutvinskiy Y, Soininen H, Zubarev R A. 2011. Alzheimer's disease and mild cognitive impairment are associated with elevated levels of isoaspartyl residues in blood plasma proteins. J Alzheimer's Dis 27, 113-118). Therefore, high occupancy of isoAsp residues in blood proteins represents a risk factor for developing AD and other neurodegenerative diseases. According to the isoAsp hypothesis of AD, the appearance of this risk factor precedes all other known AD hallmarks (FIG. 2). We also obtained evidence that, when AD is already diagnosed, high isoaspartate content in blood proteins increases the risk of fast mental decline (FIGS. 3 and 4).

Antibodies are large proteins present in blood that have the ability to bind to undesired molecules and help eliminate them from the organism. There are astronomical numbers of different antibodies present in human blood that are specific for myriad of viruses, bacteria, their toxins and undesired modifications in proteins. There are also anti-isoaspartate (anti-isoAsp) antibodies in blood that help eliminate deamidated proteins. We have found that AD patients have significantly lower levels of anti-isoAsp antibodies than healthy controls (FIG. 7), which makes them more susceptible to the damaging action of isoaspartate.

AD is the most common form of senile dementia. According to the World Health Rankings, death rate caused by AD in Sweden ranks the fourth in the world, right after Finland, Iceland, and the United States. In the year 2008 alone, 53 billion Euros were spent to care for the dementia patients in Nordic countries alone (Wimo A, Jönsson L, Gustavsson A, McDaid D, Ersek K, Georges J, Gulácsi L, Karpati K, Kenigsberg P, Valtonen H. 2011. The economic impact of dementia in Europe in 2008—cost estimates from the Eurocode project. International Journal of Geriatric Psychiatry 26, 825-832). It has been estimated that one in ten people over age of 65 and nearly half of people over 85 have AD. With the current 20 percent, and rising, fraction of pensioners in the total population, AD is a heavy burden for the Swedish national health and social care systems.

There is currently no cure for AD, and the approved drugs provide only symptomatic treatment. But while curing AD proved very difficult, its prevention is a more realistic goal. Delaying the onset of AD by 5 years would reduce the number of AD cases by half; delaying by 10 years would practically eliminate the disease as an epidemic phenomenon. In order to establish cost-effective prevention program, it is important to perform early diagnosis of AD. For the benefit of the AD patients themselves, their relatives and employers, it is also important to be able to prognosticate the AD development once it is diagnosed. Since measuring the isoAsp in blood proteins as well as the level of anti-isoAsp antibodies can help assessing the risk of AD developing, or, when AD is already diagnosed, estimate the risk of fast mental decline, it is important to create a cheap and accurate method for such measurements.

Another problem related to isoAsp content in blood is the short shelf life of human blood plasma stored in blood banks, typically 12 months. Human blood is a precious biological liquid, of which 28 million liters are collected and fractionated to obtain plasma or serum worldwide yearly. About seven percents of the collected blood are discarded immediately, mostly due to viral infection. Around 3 percent, or 1 million liters, are eventually discarded due to storage problems or expiration date. At the conditions of storage (typically, −20-25° C.), Asn deamidation still takes place, and thus the isoAsp levels builds up with storage time. It is important to measure the isoAsp content in stored blood to avoid transfusing blood with unhealthy elevated levels of isoAsp, as transfusion of such blood may carry risk of neurodegenerative disease.

Besides, some blood donors have elevated levels of isoAsp in blood proteins because of the genetic factors or their health state, and thus their blood is unsuitable for donation. A simple test measuring the isoAsp level is needed to determine whether the isoAsp level in blood with expired shelf live is still suitable for transfusion, and thus help saving thousands of litres of human blood annually. Also, such test could help reducing the risk of disease being triggered by blood transfusion.

Measuring isoasparatate residue occupancy or concentration in blood is not trivial. One approach is to use sophisticated and expensive method of liquid chromatography combined with tandem mass spectrometry (LC-MS/MS), similar to the one used in the cited work by Yang et. al. The only commercially available method, called ISOQUANT, measures the rate of enzymatic isoasparate conversion to aspartic acid by PIMT using SAM. The ISOQUANT readout is the concentration in the sample of the reaction product, SAH. To obtain the desired value of the isoaspartate concentration in proteins, the measured SAH level has to be normalized by the protein concentration, which has to be measured by a different assay. FIG. 3 shows the result of ISOQUANT analysis revealing elevated isoAsp concentration in blood proteins of 12 AD patients whose mental abilities declined fast compared to 12 slow declining patients. In FIG. 4, the receiver operating characteristic (ROC) curve obtained based on these data is shown. The area under the curve is 0.82, indicating satisfactory analytical performance in predicting the rate of mental decline of AD patients by isoAsp content in blood proteins.

However, the heterogeneous nature and high cost of the current isoaspartate analysis based on ISOQUANT or LC-MS/MS are the factors preventing broader use of such analyses in clinical practice. It would have been advantageous to utilize for that purpose a simpler, faster and more conventional biochemical assay based on antibodies.

One such assay is the enzyme-linked immunosorbent assay (ELISA), first described by Engvall and Perlmann (J Immunol 1972, 109, 129-135). ELISA is currently extremely widespread in blood analysis. However, ELISA requires anti-isoAsp antibody, and due to the low immunogenicity of isoasparate and small size of its chemical group, it is difficult to develop an antibody that would be specific for isoasparate residues in all amino acid contexts. This is the main reason why an antibody-based test for measuring isoaspartate concentration or occupancy in blood proteins had not been reported to date.

### BRIEF SUMMARY OF THE INVENTION

The present invention utilizes the fact that more than half of the protein content in blood plasma and serum is due to a single abundant protein, human serum albumin (HSA), with the molecular weight of about 67 kilodaltons. The residence time of an HSA molecule in blood is normally ≈25 days. As the HSA sequence contains several asparagine residues, during this time at least one of these residues is likely to deamidate and convert to isoaspartate. When passing through liver, the isoAsp residues undergo repair by PIMT and convert to normal Asp residue. The repair reaction has low specificity towards the protein sequence; therefore, the level of isoaspartate in HSA is on average the same as in other blood proteins.

In current invention, the isoAsp level in HSA is used as a “thermometer” measuring the general level of isoAsp in blood proteins and thus assessing the state of the isoAsp repair mechanism in a given human organism. Also, because of the HSA abundance, among the anti-isoAsp antibodies in normal blood there must be many directed against the deamidated sites in that protein. Thus deamidated HSA can serve as antigen (bait) to immobilize such antibodies, and their abundance can be used as a proxy for the general level of anti-isoAsp antibodies in a given organism.

Out of the several potential deamidation sites in HSA, the sites that are located on the protein surface are more prone to deamidation, for which access to water is important. These sites are also more accessible for the repair mechanism than the sites that are buried inside the protein. Thus these sites are most representative of the balance between deamidation and its repair in the whole blood proteome. An antibody can be created directed against these sites. Such an antibody can be used to design an ELISA for assessing the isoAsp content in these sites as a proxy for the general isoAsp content in all blood proteins.

Some of these representative deamidation sites are located in the HSA sequence between the cleavage positions for trypsin or other specific protease. Thus cleavage of HSA by these proteases can yield peptides amenable to LC-MS/MS analysis of the isoaspartate occupancy, which is percentage of Asn residues concerted to isoAsp. For such measurements one needs to detect and quantify the corresponding peptides with and without isoaspartate and calculate the ratio of their abundances. The LC-MS/MS analysis with electron transfer dissociation as in the cited paper by Yang et al. can be used as a reference method for verifying the ELISA results.

We have obtained monoclonal antibody against a peptide representing an isoAsp-containing partial sequence of HSA. Such an antibody circumvents the need for an antibody specific to all isoaspartic acid residues, which has proven very difficult. By artificial deamidation of HSA performed by incubation for 6 weeks at 60° C. and pH 8.5, we obtained a stable standard of deamidated HSA and characterized it by LC-MS/MS. This standard is used for calibration of the antibody-based assays.

The deamidated HSA was also used as an antigen to immobilize the anti-isoAsp antibodies in human blood and measure their relative abundance.

The current disclosure provides a method for assessing whether or not a given test blood sample belong to a normal group or a risk group. Depending on the definition of normal, this can be a group of people free of Alzheimer's disease and other related neurodegenerative disorders, or a group of slowly declining Alzheimer's disease patients, or a group of blood donors with acceptably low isoAsp content in blood proteins, or a group of blood samples stored in a blood bank in a frozen state and still suitable for blood transfusion. The disclosed method comprises: obtaining a first set of test blood samples and a second set of blood samples that are considered belonging to a normal (control) group; obtaining plasma from said blood samples; measuring the relative abundance of anti-isoaspartate antibodies in each plasma sample; measuring the occupancy of isoaspartate residue in a representative HSA sequence location in each plasma sample; based on the distributions in the set of normal plasma samples of the relative abundances of anti-isoaspartate antibodies and of the occupancies of isoaspartate residues in a representative HSA sequence location, establishing a statistical model for the probability for a given plasma sample to be a normal sample; attributing every plasma test sample to either normal or risk group based on the maximum likelihood according to their measured values and said statistical model.

In the above-described method, for each set of test subjects, a set of healthy subjects can be selected such that the two sets match in terms of any of the following parameters or their combination: sex, age, race, ethnicity, genotype, education, profession, lifestyle, drug intake, and smoking habits.

### BRIEF DESCRIPTION OF THE DRAWINGS

FIG. 1. Description of the mechanism of isoAsp formation from the residues of amino acids asparagine (Asn) and aspartic acid (Asp) and its repair by the enzyme PIMT using SAM as a cofactor.

FIG. 2. The time progression of the Alzheimer's disease (AD) according to the isoaspartate hypothesis of AD, starting from the healthy state, presymptomatic state, mild cognitive impairment (MCI) and clinical AD.

FIG. 3. Relative isoAsp concentrations in blood serum in the AD patient groups of fast and slow decliners. The p-value for the difference between the groups is below 0.05.

FIG. 4. ROC curve for data in FIG. 2 for differentiating between the fast and slow decliners in AD. The area under the curve is 0.82.

FIG. 5. Sequence of Human Serum Albumin and its 3D structure with the selected representative tryptic peptide highlighted.

FIG. 6. Four steps in a typical sandwich ELISA.

FIG. 7. Anti-isoAsp antibody abundance and isoAsp content in HSA in blood samples from a healthy cohort (n=20) and an age-matched AD cohort (n=20), with equal male and female participation in each cohort. The accuracy of separating the two cohorts by anti-isoAsp antibody abundances (horizontal threshold I) is 94%, and by both anti-isoAsp antibody abundances and the isoAsp content in HSA (curved threshold II) the accuracy is 97%. The p-value of the separation of two cohorts is less than 10⁻¹¹.

### DETAILED DESCRIPTION OF THE INVENTION

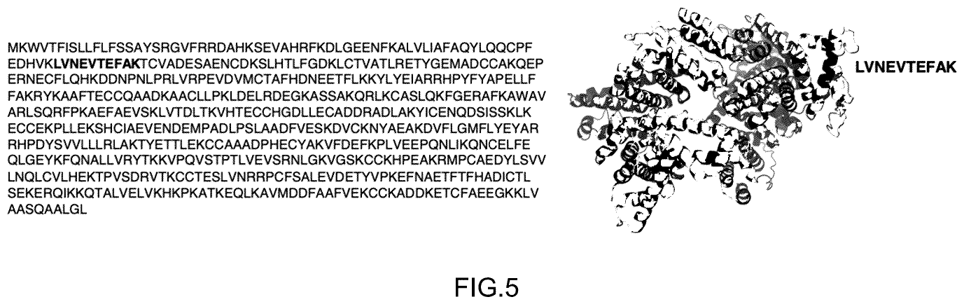

As a representative sequence in HSA that is prone to deamidation and isoAsp formation, -VNE- is chosen. The position of the representative tryptic peptide LVNEVTEFAK in the HSA sequence and the 3D HSA structure is shown in FIG. 5. Mouse monoclonal antibody was raised against the deamidated peptide LV(isoD)EVTEFAK, where (isoD) is the isoaspartate residue.

Based on this antibody, an ELISA test was created as described below.

### ELISA Production and Test Application

Antibody (or immunoglobulin, IgG) molecules are glycoproteins composed of one or more units, each containing four polypeptide chains: two identical light chains and two identical heavy chains, connected by disulphide bridges. The five primary classes of immunoglobulins are IgG, IgM, IgA, IgD and IgE. These classes are distinguished by the type of heavy chain found in the molecule. IgG molecules have heavy chains known as g-chains; IgMs have μ-chains; IgAs have a-chains; IgEs have e-chains; and IgDs have d-chains. The main function of antibodies is to recognize and bind to a molecule known as antigen. Usually the antibodies binding to an antigen protein only recognize in that protein a short amino acid sequence known as epitope. The organism typically produces many different antibodies binding to the same antigen; the mix of such antibodies is called polyclonal. Polyclonal antibodies have excellent sensitivity, but not always excellent specificity, as some antibodies in the mix may also bind to other sequences. In analytical applications, polyclonal antibodies are preferentially avoided, as the same antibody mix cannot be exactly reproduced twice. In contrast, monoclonal antibodies that are produced by genetically identical B-cells, called clones, can be produced in any desired quantities for any duration of time, as long as the clones are kept alive and devoid of mutation.

### Method to Create Mouse Monoclonal Antibody

Step 1: Immunization. First, the peptide against which an antibody is created is fused with a carrier protein. This fusion ensures long time of circulation in blood before degradation. Then mice are immunized at different antigen concentrations to have a broad spectrum of immune responses. After an incubation period, mice blood is tested to identify the animal with the best immune response to immunisation.
Step 2: Isolation of spleen cells including the antibody producing B-cells from the mouse with the best response.
Step 3: Fusion. The isolated B-cells are fused with human myeloma cells, thus obtaining antibody producing hybridoma cells with endless growth and division potential.
Step 4: Screening for hybridoma clones producing the desired antibodies at the highest rate.
Step 5: Subcloning. As the selected clone may still contain cells producing multiple antibody variants, limited dilution is performed, so that one well in a growth plate contains maximum one cell. A subsequent cultivation followed by screening ensures that the selected subclone with the best antigen recognition is used for antibody production and purification.

### Antibody Purification and Characterization

The production of larger antibody quantifies is performed by growing clone for several weeks in serum free medium. Liquid chromatography or immunoprecipitation using a protein A column or protein G column is usually employed to purify large monoclonal antibody quantities. Screening is performed for antibody specificity and affinity. Antibody isotype (class and subclass) are determined, and sequencing of the variable regions is performed.

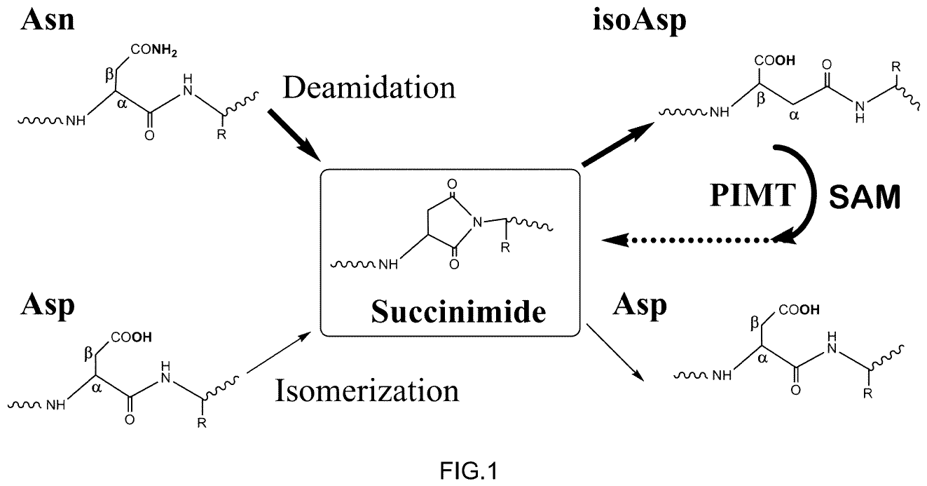

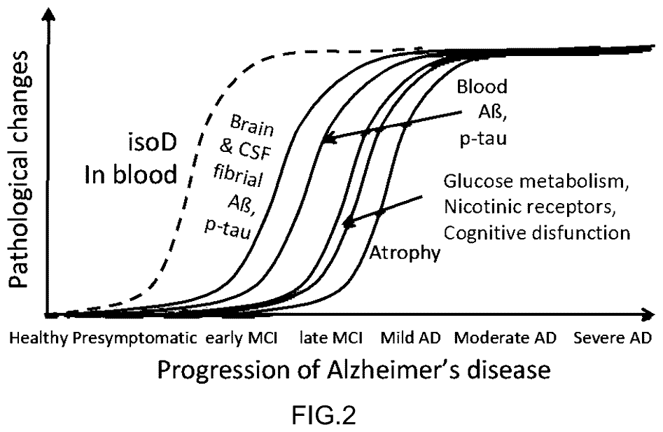

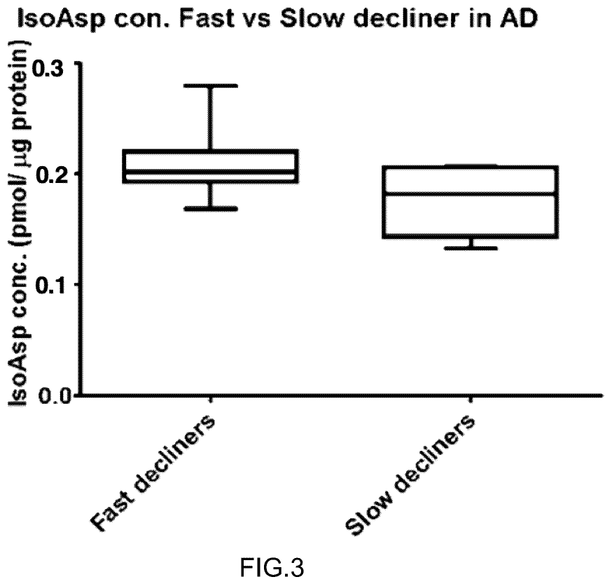

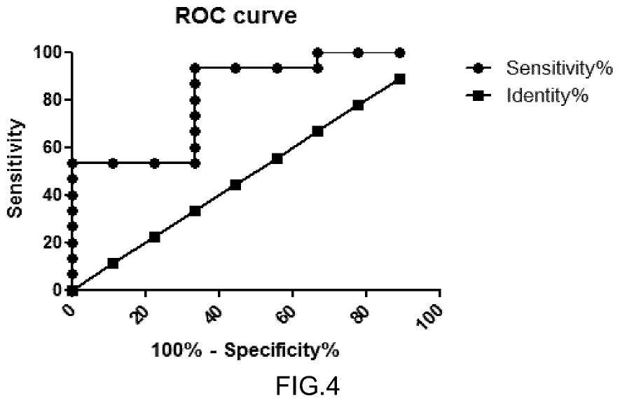

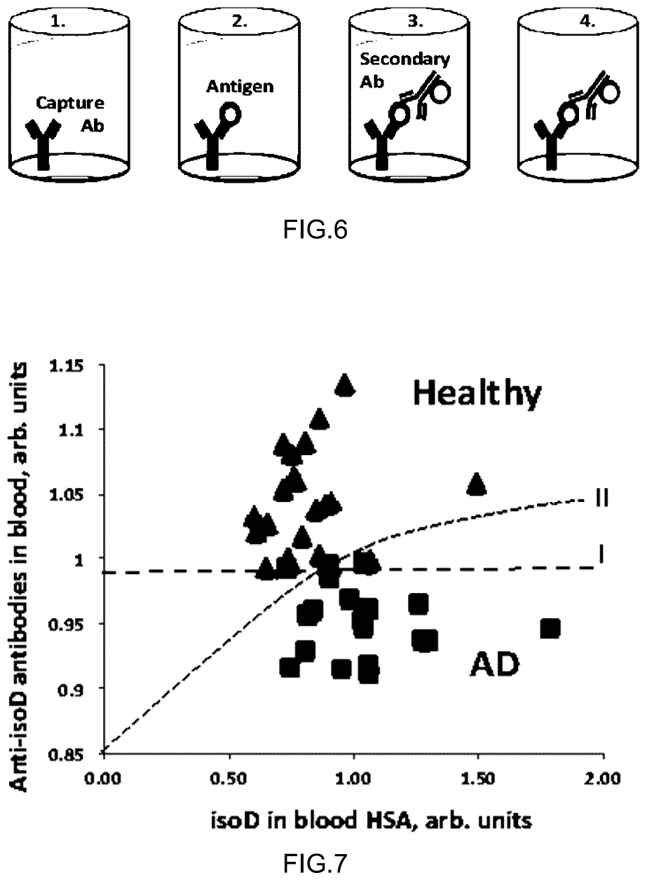

Enzyme-Linked Immuno Sorbent Assay (ELISA) is intended for quantitative or qualitative measurements of the antigen in a complex matrix. Several types of ELISAs exist, the most widely used being sandwich ELISA. Sandwich ELISA typically requires the use of matched antibody pairs, where each antibody is specific for a different, non-overlapping part (epitope) of the antigen molecule. A first antibody, known as capture antibody, coats the well bottoms (FIG. 6-1). Sample solution containing antigen is then added to the well, and the antigen binds to the capture antibody (FIG. 6-2). If the antigen is very abundant, as in the case of HSA, the first antibody is not absolutely needed, as the antigen can spontaneously deposit a monolayer on the wells and bottom of the test tube. A second antibody, known as detection antibody, is then added in order to detect the captured or deposited molecule containing the antigen (FIG. 6-3). This antibody is conjugated with an enzyme, typically horse-radish peroxidase (HRP), which catalyses the oxidation of luminol, which leads to emission of blue light (FIG. 6-4). Sometimes the secondary antibody doesn't contain the enzyme, and then a third detecting antibody conjugated with an enzyme is used. In this case, the third antibody has to be specific to the secondary antibody but not the capture antibody. Therefore, the latter two antibodies have to originate from different organisms.

### Example 1

### Blood Sample Collection and Preparation of Plasma and Serum

The blood sample is taken after fasting between 7 and 10 o'clock in the morning and 2-10 mL of whole blood are collected into commercially available anticoagulant-treated tubes. Blood cells are removed from plasma by centrifugation for 10 min at 1,000-2,000×g using a refrigerated to 2-8° C. centrifuge. Centrifugation for 15 min at 2,000×g depletes platelets in the plasma sample. The resulting supernatant is designated plasma. A 20-min sonication procedure is applied to break down the aggregates and solubilize the released HSA molecules. And the clot and other insoluble proteins are removed by centrifuging at 20,000×g for 10 min at 2-8° C. The resulting supernatant is immediately transferred into a clean polypropylene tube using a Pasteur pipette.

If not analyzed immediately, the sonicated plasma should be apportioned into 0.5 mL aliquots, stored, and transported at −20° C. or lower. It is important to avoid freeze-thaw cycles. The collected samples should be stored at −80° C. and transferred on dry ice.

### ELISA Procedure

In the preferred implementation, sample of plasma is diluted by Phosphate-buffered saline (PBS) solution to concentration of 10 μg/mL. Then each well in a Pierce 96-well white opaque polystyrene plate is coated with 50 μL of the sample solution by incubating at room temperature for 2 hours. The wells are then washed three times with 300 μL of the PBST buffer containing 0.05% solution of Tween-20 (Sigma Aldrich) in PBS, with the liquid being removed by flapping the plate on tissue. Each well is then covered with 50 μL of the locking buffer, containing 10% milk powder dissolved in PBST. After that each well is washed three times with 300 μL of PBST and incubated overnight at 4° C. with 200 μL Pierce blocking buffer. Then the stock solution of the anti-isoAsp antibody is diluted by blocking buffer to concentration of 800 ng/ml, 50 μL of such solution is added to each well, and left to incubate for 2 hours at room temperature, after which the wells are washed three times with 300 μL of PBST. The stock solution of goat anti-mouse IgG antibody conjugated with the enzyme horseradish peroxidase in glycerol, 1:1 v/v, is diluted by blocking buffer at a 1:5000 ratio, and 50 μL of the diluted solution is added to each well and incubated for 2 hours at room temperature. After that to each well is added, as quickly as possible, 100 μL of the ELISA chemiluminescent substrate working solution containing luminol enhancer and peroxide solution. The chemiluminescence from the plate is then immediately analyzed by a detection imager.

Preparing Artificially Deamidated HSA with High isoAsp Occupancy

In the preferred implementation, 5 mg Human Serum Albumin were incubated in 1 mL Tris pH 8.5 buffer at 60° C. for 42 days. The LC-MS/MS analysis of the representative tryptic peptide LVNEVTEFAK from the HSA digest showed 60% isoAsp occupancy in the -VNE- position.

Performing isoAsp Occupancy Measurements in Sample

The calibration curve for the isoAsp occupancy scale is obtained by serial dilution of the calibrator solution containing deamidated HSA in fresh HSA solution and performing ELISA. The calibration curve is an interpolated dependence between the measured ELISA signal and the known average isoAsp occupancy in the isoAsp-containing calibrator. Comparison of the ELISA signal from test samples with the calibration curve provides the isoAsp occupancy in percent (%) in test samples.

### Performing Anti-isoAsp Antibody Measurements in Blood Samples

In the preferred implementation, a sample of the artificially deamidated HSA is diluted by PBS to a final concentration of 10 μg/mL, and then 100 μL are incubated in each well of the 96-well plate for 2 hours before removing and washing three times with 300 μL of PBST. From each blood sample, antibodies are extracted by Melon™ gel extraction (Thermo Scientific), and their concentration is adjusted to 2 μg/mL by diluted PBS. For statistically reliable measurements, the extraction needs to be performed in several (n>3) replicates. The extracted antibodies are incubated for 2 hours at room temperature in the 96-well plate with the deamidated HSA adsorbed on the surfaces after which the wells are washed three times with 300 μL of PBST. The stock solution of goat anti-mouse IgG antibody conjugated with the enzyme horseradish peroxidase in glycerol, 1:1 v/v, is diluted by blocking buffer at a 1:5000 ratio, and 50 μL of the diluted solution is added to each well and incubated for 2 hours at room temperature. After that to each well is added, as quickly as possible, 100 μL of the ELISA chemiluminescent substrate working solution containing luminol enhancer and peroxide solution. The chemiluminescence from the plate is then immediately analyzed by a detection imager. If a multi-channel pipette is used for sample preparation, readout can be normalized by the average measurements in channel each to eliminate the effect of channel-to-channel variation. The normalized values are then taken as relative abundances of the anti-isoAsp antibodies.

### Attributing Test Sample to Either Normal or Risk Group

In one implementation, the isoAsp occupancy in HSA and the relative abundance of anti-isoAsp antibodies from healthy individuals as well as individuals with a neurological disorder are measured, and a thresholds for each type of measurements is determined between the two groups such as to obtain the highest accuracy of classification, determined as the percentage of the samples classified correctly. The individuals with below-threshold isoAsp occupancy in HSA and abundance of anti-isoAsp antibodies are classified as healthy, while the individuals with isoAsp occupancy in HSA and abundance of anti-isoAsp antibodies above the threshold and are classified as belonging to the risk group. As an example, FIG. 7 shows the isoAsp ELISA and anti-isoAsp antibody results of blood samples from healthy cohort (n=20, average age 63 years) and cohort with AD (n=20, average age 64 years), with equal male and female participation in each cohort. The threshold at 0.99 arbitrary units for anti-isoAsp antibodies (threshold I) identifies correctly 18 out of 20 healthy individuals as well as 19 out of 20 AD patients, reaching the classification accuracy of 37/40=92.5%. The use of the isoAsp content in HSA in addition to the relative abundance of anti-isoAsp antibodies (curved threshold II) identifies correctly 19 healthy individuals and 19 AD patients, reaching the classification accuracy of 28/40=95%.

What is claimed is:

1. (canceled)

2. (canceled)

3. (canceled)

4. (canceled)

5. (canceled)

6. (canceled)

7. (canceled)

8. (canceled)

9. A method aiming to determining whether a given test human blood sample belongs to a person at risk of developing Alzheimer's disease or another related neurodegenerative disorder or at risk of fast progress of diagnosed Alzheimer's disease or another related neurodegenerative disorder, a method comprising:
obtaining a set of normal blood samples from persons without symptoms of a neurodegenerative disease;
obtaining plasma from said blood samples and measuring in them isoaspartate occupancy in an abundant blood protein and relative abundance of anti-isoaspartate antibodies;
establishing a statistical model for the probability for a given isoaspartate occupancy in an abundant blood protein and relative abundance of anti-isoaspartate antibodies to originate from a normal sample by fitting Gaussian distributions to the distributions of the isoaspartate occupancies in an abundant blood protein and relative abundances of anti-isoaspartate antibodies obtained for the set of normal plasma samples;
calculating for a given test human blood sample the probability to belong to the normal group based on said statistical model and measured isoaspartate occupancy in an abundant blood protein and relative abundance of anti-isoaspartate antibodies, with the obtained probability lower than a defined threshold, such as 0.05 or lower, indicating the presence of said risk of neurodegenerative disorder.

10. The method of claim 9, in which the relative abundance of anti-isoaspartate antibodies in human blood is measured using an immunochemistry assay with an artificially deamidated human serum albumin as an antigen.

11. The method of claim 9, in which the occupancy of isoaspartate is measured in human serum albumin.

12. The method of claim 11, in which the occupancy of isoaspartate (iso Asp) is measured in the sequence location -LV(isoAsp)EV-.

13. The method of claim 9, in which the normal set of samples is selected such that it matches the test blood sample in terms of any of the following parameters or their combination: sex, age, race, ethnicity, genotype, education, profession, lifestyle, drug intake, smoking habits.

14. The method of claim 12, in which the occupancy of isoaspartate residue isoAsp in the sequence location -LV(isoAsp)EV- in human serum albumin is measured with help of a molecule specifically binding to that sequence location, the molecule that can be a protein, an antibody, an RNA, a DNA, or a small organic molecule.

15. The method of claim 14, in which the specifically binding molecule is a monoclonal antibody with a variable sequence that is not different by more than one amino acid from the following variable sequence:
Heavy chain, VH:
EVKLVESGGGLVQPGGSLRLSCATSGFTFTDHYMSWVRQPPGK
ALEWLGFIRNKANGYTTEYSASVKGRFTISRDNSQSILYLQMNT
LRAEDSATYYCARDKGGYDPWFAYWGQGTLVTVSA
Light chain, VL:
DIQMNQSPSSLSAYLGDTITITCHASQNINVWLSWYQQKPGNIP
KLLIYKASNLHTGVPLRFSGSGSGTGFTLTISSLQPEDIATYYCQ
QGQSYPLTFGAGTKLELK.


SEQUENCE LISTING (2 sequences)

SEQ ID NO: 1 - protein, 122 aa, Mus musculus
EVKLVESGGGLVQPGGSLRLSCATSGFTFTDHYMSWVRQPPGKALEWLGFIRNKANGYTTEYSASVKGRFTISRDNSQSILYLQMNTLRAEDSATYYCARDKGGYDPWFAYWGQGTLVTVSA

SEQ ID NO: 2 - protein, 107 aa, Mus musculus
DIQMNQSPSSLSAYLGDTITITCHASQNINVWLSWYQQKPGNIPKLLIYKASNLHTGVPLRFSGSGSGTGFTLTISSLQPEDIATYYCQQGQSYPLTFGAGTKLELK